(19) United States
(12) Patent Application Publication
(10) Pub. No.: US 2023/0320925 A1
(43) Pub. Date: Oct. 12, 2023

(54) METHODS AND DEVICES FOR MANIPULATING THE GLYMPHATIC SYSTEM FOR MEDICAL TREATMENTS

(71) Applicant: David Smith, West Chester, OH (US)

(72) Inventor: David Smith, West Chester, OH (US)

(21) Appl. No.: 18/297,067

(22) Filed: Apr. 7, 2023

Related U.S. Application Data
(60) Provisional application No. 63/362,688, filed on Apr. 8, 2022.

(51) Int. Cl. A61H 9/00; A61M 16/00; A61M 16/06

(52) U.S. Cl. CPC A61H 9/0092; A61M 16/0045; A61M 16/0683; A61H 9/0007; A61H 2201/107; A61H 2205/04; A61H 2201/1609; A61H 2201/165; A61H 2201/1676; A61H 2201/1246; A61H 2201/1215

(57) ABSTRACT

Methods and devices are provided for manipulating the glymphatic system to improve cognitive and other cranial or brain functions of a subject. In particular, pressure is alternately applied to the jugular veins on either side of the subject's neck to generate a to and fro movement of cranial fluids.

### PRIORITY CLAIM

This application is a utility filing from and claims priority to U.S. Provisional Application No. 63/362,688, filed on Apr. 8, 2022, the entire disclosure, including appendices, of which is expressly incorporated herein by reference.

### BACKGROUND

This invention relates generally to methods and devices for improving cognitive and other brain functions by facilitating the Glymphatic System. The Glymphatic System has only recently been described and named. See, Iliff J J, Wang M, Liao Y, Plogg B A, Peng W, Gundersen G A, et al. (August 2012). “A paravascular pathway facilitates CSF flow through the brain parenchyma and the clearance of interstitial solutes, including amyloid β”. Science Translational Medicine. 4 (147). The Glymphatic System is a waste clearance system that utilizes a unique system of peri-vascular channels, formed by astroglial cells, to promote efficient elimination of soluble proteins and metabolites from the central nervous system of vertebrates. See, Benveniste et al., “The Glymphatic System and Waste Clearance with Brain Aging: A Review”, Gerentology 2019:65:106-119 (Jul. 11, 2018). Besides waste elimination, the glymphatic system may also function to help distribute non-waste compounds, such as glucose, lipids, amino acids, and neurotransmitters related to volume transmission, in the brain. This system involves the circulating and transporting of cerebro-spinal fluid (CSF) along the peri-arterial spaces, followed by convective flow through the brain parenchyma (or down the spinal column), and exit of interstitial fluid (ISF) along the peri-venous space to the cervical lymph system. This appears to be an energy-requiring process that is driven by multiple mechanisms, thus improving alertness and displacing somnogenic substances from the brain. The system may have utility in helping the brain to augment the repair process in many traumatic, infectious or debilitating disorders. Lastly it could also improve drug delivery or even help to reduce brain aging.

### Definitions

Attention—The mental ability to select stimuli, responses, memories, and thoughts that are behaviorally relevant among a host of others that are behaviorally irrelevant. Attention does not imply a singular mechanism; rather, it is a complex system presiding over a number of distinct neuronal circuits.

Attentional Network—Neural circuits subservient to attentional processing, which preserve a degree of anatomical and functional independence but interact in many practical situations

Executive Network—The mechanism for monitoring and resolving conflict among thoughts, feelings, and responses; an attentional system concerned with such tasks as working memory, planning, switching, and inhibitory control

Orienting—The process of selecting information from sensory input

Alerting—The process of achieving and maintaining a state of high sensitivity to incoming stimuli. Alerting involves a change in the internal state in preparation for perceiving a stimulus. The alert state is critical for optimal performance in tasks involving higher cognitive functions.

Cognitive Control—Processes such as conflict resolution, error correction, inhibitory control, planning, and resource allocation

Fluid systems within the cranium—Arterial and venous blood, Cerebral Spinal Fluid, and interstitial (regions between cells) fluids

Somnogenic Substances—any endogenous factor that, at elevated levels in the CSF and/or brain parenchyma, induces somnolence. Somnogenic substances include, for example, prostaglandin D2 (PGD2) and adenosine

### SUMMARY OF THE DISCLOSURE

In one aspect, the invention provides a method for improving a brain function in a subject by increasing intracranial pressure and volume, diverting cranial fluid volumes, or both. Brain functions that may be improved include, for example, arousal, attention/attentiveness, executive functions, learning, memory, motor coordination, spatial awareness, and vigilance. Also, many dementive or debilitating brain disorders (even aging) could be improved.

In some embodiments, the intracranial pressure (ICP), intracranial volume (ICV), or both, is increased by diverting flow to the venous capacitance vessels of the brain by reducing the jugular venous outflow. Cranial venous flow may be diverted by applying pressure to one or more (e.g., one, two, three, four, or more) neck veins including, for example, the internal jugular vein(s) (“IJVs”) and the external jugular vein(s) (“EJVs”). This flow is then diverted to the venous capacitance vessels and vertebral veins and as the vessels dilate, the cerebral spinal fluid (CSF) equilibrates by circulating down through the interstitial and spinal canal spaces (defining the Glymphatic System). Pressure may be applied to any neck vein unilaterally, bilaterally, and/or in pairs, but optimally, in an intermittent manner allowing a “to and fro” action upon compression. For example, in some embodiments, pressure is applied to both IJVs, both EJVs, one IJV and one EJV, or all four veins simultaneously, but importantly, the pressure is released and reapplied in a cyclical manner and usually alternating from right to left or left to right.

In some embodiments, a cyclic compression and release of the above neck veins is undertaken to better effectuate flow through the Glymphatic System.

In yet another embodiment, this cyclic compression and release is undertaken to facilitate flow through the act of dilating and deflating the petrosal sinuses (residing within the petrosal sulcus) with the intent to improve flow through the eye vasculature or even promote flow to or from the pituitary gland.

In yet another embodiment, this cyclic compression and release is undertaken to facilitate flow through the act of dilating and deflating the endolymphatic sac with the intent to improve flow through the auditory and vestibular apparatus to potentially improve Meniere's disease.

The frequency of these cyclic compressions and releases discussed above is undertaken at fast, moderate or slow rates. The fastest rate would be measured in cycles per minute, the moderate rate would be measured in cycles per hour and the slowest in cycles per day.

In a second application, cerebral fluid flows (The Glymphatic System) can be facilitated by manipulation of the body's carbon dioxide levels often measured as end-tidal CO(ETCO₂).

In another embodiment, the CO₂ levels, can be manipulated by adding canister derived (exogenous) CO₂, or even more importantly, by rebreathing or recycling one's own exhaled breaths (endogenous) to attain specific predetermined ETCO₂ levels. As CO₂ is the number one determinant of intracranial arterial flow, it results in the ability to modulate the resultant intracranial volume and pressure as well.

As CO₂ increases the cerebral arterial flow rate, it results in changes in the Intracranial Volume and Pressure and thus takes up or reduces the Compensatory Reserve Volume of the cranial space and moves to equilibrate, the Cerebral Spinal Fluid (which has the path of least resistance) and interstitial fluids flow downward (and actually dilating the spinal bulb) and out of the cranial-spinal space (again defining The Glymphatic System).

In another aspect, the invention (embodied by jugular compression and/or manipulating CO₂) provides a method for reducing the concentration of somnogenic substances in the brain of a subject (e.g., the brain parenchyma including the cortical regions) by reducing the jugular cranial venous outflow (or altering the CO₂). Cranial jugular venous outflow may be diverted by applying pressure to one or more (e.g., one, two, three, four, or more) neck veins including, for example, the internal jugular vein(s) (“IJVs”) and the external jugular vein(s) (“EJVs”). Pressure may be applied to any neck vein unilaterally, bilaterally, and/or in pairs. For example, in some embodiments, pressure is applied to both IJVs, both EJVs, one IJV and one EJV, or all four veins simultaneously. CO₂ levels can be manipulated by altering the exogenous or endogenous CO₂ inspiration levels.

In some embodiments of the foregoing aspects, the neck vein pressure is at least about 10, 20, 30, 40, 50 mm Hg or more and/or less than about 60, 70, 80, 90, or 100 mm Hg.

In some embodiments of the foregoing aspects, the neck vein pressure is applied by a device worn on or about the neck of the subject.

In some embodiments of the foregoing aspects, the neck vein pressure is applied continuously. Optionally, the neck vein pressure is applied for at least 0.5, 1, 5, 10, 15, 30, or 60 minutes, or at least 2, 4, 6, 8, or 10 hours or more, depending upon the treatment protocol. In some embodiments, the neck vein pressure is at least about 10, 20, 30, 40, 50 mm Hg or more and/or less than about 60, 70, 80, 90, or 100 mm Hg.

In some embodiments of the foregoing aspects, the neck vein pressure is applied intermittently. Optionally, the intermittent pressure application substantially has a two-stage cycle characterized by an on-time having a first occlusive neck vein pressure, and an off-time having a second occlusive neck vein pressure that is lower than the first occlusive pressure. In some embodiments, the difference between the first occlusive pressure and the second occlusive pressure is at least about 10 mm Hg, 15 mm Hg, 20 mm Hg, 25 mm Hg, 30 mm Hg, 35 mm Hg, 40 mm Hg, 45 mm Hg, 50 mm Hg, 55 mm Hg, 60 mm Hg, 65 mm Hg, 70 mm Hg, 75 mm Hg, or 80 mm Hg. In some embodiments, the first occlusive pressure is at least about 10, 20, 30, 40, 50 mm Hg or more and/or less than about 60, 70, 80, 90, or 100 mm Hg. In some embodiments, the second occlusive pressure is about 0 mm Hg, 5 mm Hg, 10 mm Hg, 15 mm Hg, 20 mm Hg, 25 mm Hg, 30 mm Hg, 35 mm Hg, or 40 mm Hg.

In some embodiments of the foregoing aspects, the on-time of at least one cycle, or preferably each cycle, is at least about 1, 2, 3, 4, 5, 10, 15, 30, 45, or 60 seconds, or at least about 2, 3, 4, 5, 10, 15, 30, or 60 minutes, or at least about 2, 3, 4, or 5 hours or more. In other embodiments, the off-time of at least one cycle, or preferably each cycle, is at least about 1, 2, 3, 4, 5, 10, 15, 30, 45, or 60 seconds, or at least about 2, 3, 4, 5, 10, 15, 30, or 60 minutes, or at least about 2, 3, 4, or 5 hours or more. In some embodiments, the on-time of each cycle is between about 1 second and 5 minutes. In some embodiments, the off-time of each cycle is between about 1 second and 5 minutes. In some embodiments, each on-time and each off-time of each cycle is between about 1 second and 5 minutes.

In some embodiments of the foregoing aspects, the method applies neck vein pressure intermittently for at least 2, 5, 10, 15, 20, 25, 50, 75, 100, 150, 200, 250, or more complete cycles.

In some embodiments of the foregoing aspects, the subject is engaged in a test-taking activity. In some embodiments, the test assesses locomotor coordination, activity and/or acuity, memory, learning, reasoning, or problem-solving.

In some embodiments, the subject is not at risk of receiving a concussive or traumatic force to the head or body that may otherwise result in a concussion, sub-concussive injury, or other physical injury to the brain and/or spinal cord. Rather, the subject may be at risk of Glaucoma or even Spaceflight Associated Neuro-ocular Syndrome (SANS), conditions which both threaten the individual's eyesight,

In another aspect, a device is provided that is configured to be worn around the neck of a subject and adapted to apply intermittent pressure to one or more (e.g., one, two, three, four, or more) neck veins including, for example, the internal jugular vein(s) (“IJVs”) and the external jugular vein(s) (“EJVs”). The device may be configured to apply pressure to any neck vein unilaterally, bilaterally, and/or in pairs. For example, in some embodiments, the device is configured to apply pressure to both IJVs, both EJVs, one IJV and one EJV, or all four veins simultaneously.

In some embodiments, the device is adapted for an intermittent pressure application that has a two-stage cycle characterized by an on-time having a first occlusive neck vein pressure, and an off-time having a second occlusive neck vein pressure that is lower than the first occlusive pressure. In some embodiments, the difference between the first occlusive pressure and the second occlusive pressure is at least about 10 mm Hg, 15 mm Hg, 20 mm Hg, 25 mm Hg, 30 mm Hg, 35 mm Hg, 40 mm Hg, 45 mm Hg, 50 mm Hg, 55 mm Hg, 60 mm Hg, 65 mm Hg, 70 mm Hg, 75 mm Hg, or 80 mm Hg. In some embodiments, the first occlusive pressure is at least about 10, 20, 30, 40, 50 mm Hg or more and/or less than about 60, 70, 80, 90, or 100 mm Hg. In some embodiments, the second occlusive pressure is about 0 mm Hg, 5 mm Hg, 10 mm Hg, 15 mm Hg, 20 mm Hg, 25 mm Hg, 30 mm Hg, 35 mm Hg, or 40 mm Hg.

In some embodiments, the on-time of at least one cycle, or preferably each cycle, is at least about 1, 2, 3, 4, 5, 10, 15, 30, 45, or 60 seconds, or at least about 2, 3, 4, 5, 10, 15, 30, or 60 minutes, or at least about 2, 3, 4, or 5 hours or more. In other embodiments, the off-time of at least one cycle, or preferably each cycle, is at least about 1, 2, 3, 4, 5, 10, 15, 30, 45, or 60 seconds, or at least about 2, 3, 4, 5, 10, 15, 30, or 60 minutes, or at least about 2, 3, 4, or 5 hours or more. In some embodiments, the on-time of each cycle is between about 1 second and 5 minutes. In some embodiments, the off-time of each cycle is between about 1 second and 5 minutes. In some embodiments, each on-time and each off-time of each cycle is between about 1 second and 5 minutes.

In some embodiments, the device is adapted to apply neck vein pressure intermittently for at least 2, 5, 10, 15, 20, 25, 50, 75, 100, 150, 200, 250, or more complete cycles.

In some embodiments, the endpoint goal of CO₂ manipulation would result is a minimum pCO₂ rise of 1 mmHg level to a maximum rise of 25 mmHg.

In some embodiments, the rise in pCO₂ would be by way of adding columns of dead-space rebreathing using volumes of added dead-space between 25 ml up to a maximum of 2500 ml of air.

By “brain function” is meant any function of the conscious or sleeping brain, whether or not under voluntary control of the subject. Brain functions include, but are not limited to, arousal, attention/attentiveness, executive functions, learning, memory, motor coordination, spatial awareness, and vigilance. It is understood that these brain functions themselves are not discrete neurological/neurophysiological processes but instead may encompass several and overlapping functions and states.

By “arousal” is meant the general physiological and neurological state of wakefulness and perception. Thus, a state of increased or heightened arousal is associated with an elevated state of consciousness away from somnolence. “Arousal” is a general term that encompasses several distinct states or neurophysiological functions including, but not limited to, consciousness, attention, alertness, sensory perception, and thought (planning, problem solving, etc.)

By “attention” or “attentiveness” is meant the neurophysiological process of selective concentration or allocation of mental resources on a discrete physical or mental aspect (e.g., object, physical and/or mental task, etc.) to the total or partial exclusion of other aspects. Attention is related to cognitive inhibition in that the latter described the cognitive ability of filter out (eliminate or deemphasize) stimuli that are irrelevant to the aspect to which the attention is focused.

By “executive function” is meant a set of conscious cognitive processes involved in regulating behavior. Executive functions include, but are not limited to, decision making, attention, planning, reasoning, problem-solving, working memory, and cognitive inhibition.

By “spatial awareness” is meant the set of conscious and unconscious mental processes that result in a subject's understanding of its orientation and body position relative to itself and/or external aspects.

By “vigilance” is meant the ability of a subject to maintain attention over a prolonged period of time. Vigilance may be generally described in two neurological dimensions: arousal (a high degree of wakefulness) and attention (a high degree of selective concentration). The temporal component may be measured in absolute terms (e.g., extending many minutes or hours) or relative terms (a prolonged period of time relative to task at-hand).

By “somnogenic substances” is meant any endogenous factor that, at elevated levels in the CSF and/or brain parenchyma, induces somnolence. Some 50 substances have been identified and include, for example, prostaglandin D2 (PGD2) and adenosine.

By “upper ventricular cerebrospinal fluid” or “uvCSF” is meant the CSF contained with the upper ventricular system within the brain (i.e., the lateral ventricles and the third ventricle).

By “lower ventricular cerebrospinal fluid” or “1vCSF” is meant the CSF contained within the lower ventricular system (i.e., cerebral aqueduct and fourth ventricle) and the central canal of the spinal cord.

By “on-time” is meant the duration of time in which a fully- or partially-occlusive pressure is applied to one or more neck veins in a manner sufficient to increase the intracranial blood pressure and/or volume.

By “off-time” is meant the duration of time in which no occlusive pressure, or a reduced occlusive pressure, is applied to the neck veins.

### BRIEF DESCRIPTION OF THE DRAWINGS

FIG. 1 is pictorial view of the device disclosed herein worn on the neck of a human subject.

FIGS. 2A and 2B are top views of a compression device according to one embodiment of the present disclosure, shown with the left and right compression elements alternating between neutral and compression positions.

FIGS. 3A and 3B are top views of a compression device according to a second embodiment of the present disclosure, shown with the left and right compression elements alternating between neutral and compression positions.

FIGS. 4A and 4B are top views of a compression device according to a third embodiment of the present disclosure, shown with the left and right compression elements alternating between neutral and compression positions.

FIGS. 5A, 5B, and 5C are top views of a compression device according to a fourth embodiment of the present disclosure, shown with the left and right compression elements in a neutral position and alternating between neutral and compression positions.

FIGS. 6A, 6B, and 6C are top views of a compression device according to a fifth embodiment of the present disclosure, shown with the left and right compression elements in a neutral position and alternating between neutral and compression positions.

FIGS. 7A, 7B, and 7C are top views of a compression device according to a sixth embodiment of the present disclosure, shown with the left and right compression elements in a neutral position and alternating between neutral and compression positions.

FIGS. 8A, 8B, and 8C are top views of a compression device according to a seventh embodiment of the present disclosure, shown with the left and right compression elements in a neutral position and alternating between neutral and compression positions.

FIGS. 9A, 9B, and 9C are top views of a compression device according to an eighth embodiment of the present disclosure, shown with the left and right compression elements in a neutral position and alternating between neutral and compression positions.

FIGS. 10A, 10B, and 10C are top views of a compression device according to a ninth embodiment of the present disclosure, shown with the left and right compression elements in a neutral position and alternating between neutral and compression positions.

### DETAILED DESCRIPTION

The present invention provides methods and devices for improving brain functions (e.g., arousal, attention/attentiveness, executive functions, learning, memory, motor coordination, spatial awareness, and vigilance), or even physical repair of cerebral/spinal structures by altering CSF flow within the brain. CSF flow may be altered by increasing the venous pressure and/or volume within the brain or the cranium as described herein. It is understood that the CSF volume within the brain may transiently increase and/or decrease (thereby altering the CSF flow) under conditions of increased venous pressure and/or volume within the brain or the cranium.

Mild somnolence may be caused by a variety of activities or conditions including long periods of sitting or otherwise being sedentary, performing repetitive or boring (mentally unchallenging) tasks, and/or for a lack of sleep or following long periods wakefulness. It is believed that a subject experiences temporary and reversible decline in these brain functions and/or mild somnolence as the brain accumulates somnogenic substances in the uvCSF and/or the brain parenchyma, particularly in the cortical regions. The reduction in higher brain functioning and/or mild somnolence often precedes drowsiness and sleep. Somnogenic substances believed to accumulate in the uvCSF include, for example, various cytokines, prostaglandins including prostaglandin D(2), adenosine, and others. Thus, brain function may be improved by displacing or removing the somnogenic substances from within the brain parenchyma and/or the uvCSF.

It is believed that somnogenic substances are in a rapid equilibrium between the brain parenchyma and the uvCSF. Thus, parenchymal concentrations of somnogenic substances may be rapidly reduced by reducing the uvCSF concentration of those substances. This may be achieved by altering the flow of CSF within the ventricular system including by: (i) reducing the uvCSF volume, thereby reducing the total amount of somnogenic substances present within the brain; and/or (ii) mixing the uvCSF with 1vCSF. It is believed that the 1vCSF does not accumulate somnogenic substances to the same extent as the uvCSF during somnolence. Mixing 1vCSF with uvCSF would effectively dilute the concentration of somnogenic substances within the uvCSF causing a concomitant reduction in the parenchymal somnogenic substance concentration.

CSF flow may be beneficially altered within the brain by increasing the venous pressure and/or blood volume within the brain and/or cranium. This may be achieved by partially or completely impeding blood flow through one or more cranial neck veins including, for example, the internal jugular veins and/or external jugular veins. It is believed that partial or total occlusion of one, two, three, or four of the jugular veins causes a redirection of venous blood flow into the vertebral vein(s) and venous capacitance veins while filling the cranial reserve volume and thereby increasing the blood volume and/or blood pressure within the brain. It is believed that the cranial reserve volume is filled within about <1 second to about 3 seconds, or the time taken for about 1-5 heart beats, following the full or partial occlusion of one, two, three, or four cranial neck veins.

Cranial venous blood flow may be impeded by applying pressure to the tissue overlying the targeted neck vein(s). The pressure may be applied by any suitable device that has the ability, and is configured to, apply a constant or an intermittent pressure to targeted neck veins, wherein the pressure is sufficient impede the flow of venous blood egressing from the cranium. Suitable devices include, for example, a fully-circumferential or partially-circumferential collar described in U.S. Pat. Nos. 9,168,045 and 9,173,660, and patent publications U.S. 2014/0142616, 2014/0343599, WO 2012/054262, WO 2013/055409, and WO 2015/200672, each of which is hereby incorporated by reference in its entirety. Other devices and systems suitable for applying neck vein pressure are disclosed in U.S. Pat. No. 8,900,169 and WO 2014/143853, each of which is hereby incorporated by reference in its entirety.

Without being bound by any theory, preliminary observations indicate that increasing the cranial venous pressure and/or volume by jugular vein compression has a transient and a permanent effect during the venous compression period. Upon initial internal jugular vein (IJV) compression, it is believed that the cerebral venous blood is diverted from the IJV into the venous capacitance vessels of the cranium and the vertebral venous system. This causes a mass effect or “crowding” of the CSF, resulting in a ventricular constriction and movement of CSF causally and leading to an initial decrease in the uvCSF volume. This observation may result from the fact that the CSF compartment has less resistance to flow than the (venous and arterial) blood compartment. This movement of CSF flow causes a flow of the concentration of somnogenic substances within the uvCSF downward, caudally and out of the cranio-spinal space.

Following the relative cranial venous blood diversion, a new cranial pressure/volume equilibrium between the blood and CSF compartments is reached. The elevated cranial blood pressure and/or volume caused by persistent occlusion of the jugular vein(s) fills the cranial blood reserve volume and further diminishes the CSF volume. The uvCSF volume therefore is reduced as the uvCSF is pushed into the lower ventricular structures and ultimately in the central canal of the spinal cord. The CSF again flows—in a downward direction—because it has a lower resistance to flow than the venous blood. The second and more persistent equilibrium phase lasts for the duration of the venous compression and is characterized by a reduction in the overall uvCSF volume.

Accordingly, the fully- or partially-occlusive neck vein pressure may be applied continuously or intermittently/repetitively to improve brain function, depending upon the specific circumstances of the application and the effect that is desired. The continuous application of neck vein pressure (i.e., an indefinite on-time) produces the benefit of reducing the uvCSF concentration of somnogenic substances, and concomitant improvement of brain function, while being relatively easy to administer with the use of a passive device (e.g., a neck collar or other device as described above). Such a device beneficially maintains a smaller uvCSF volume for a prolonged period of time.

Devices that intermittently and repetitively apply fully- or partially-occlusive neck vein pressure may be useful in applications having a prolonged duration (e.g., 1, 2, 3, 4, 5, 6, 7, 8, 9, 10 hours, or more), and/or when extremely heightened brain function is desired. Such a device would cycle the occlusive neck vein pressure in an on/off manner, as described below. During each on/off cycle, (i) the uvCSF (high in somnogenic substances) would be mixed with the 1vCSF (low in somnogenic substances) during the first, upward/expansion phase, (ii) the total volume of uvCSF (with a lower somnogenic substance concentration) would be reduced, thereby reducing the total amount of somnogenic substances in the uvCSF compartment, and (iii) mixed again with 1vCSF during the pressure normalization of the uvCSF compartment as the occlusive pressure is removed and reapplied to begin the cycle again.

As noted above, preliminary studies indicate that cranial blood pressure and/or volume increase within about <0.5-1.0 seconds following jugular vein occlusion; or about the time taken for about 1-2 heartbeats. It is expected that normalization of cranial blood pressure and/or volume following restoration of full venous flow (i.e., removal of the occlusive pressure) takes an approximately equal time. Thus, suitable methods and devices will apply neck vein pressure with an on-time of at least one second (e.g., at least 1, 2, 3, 4, 5, 10, 15, 30, 60, 120, or 180 seconds or more) and/or will have an off-time of at least one second (e.g., at least 1, 2, 3, 4, 5, 10, 15, 30, 60, 120, or 180 seconds or more). It is understood that the on-time and off-time need not have the same duration, and that the on-time and off-time need not be the same in every cycle. It is further understood that the off-time need not return the occlusive neck vein pressure to zero. It is sufficient that the neck vein pressure is reduced during the off-time only to an amount sufficient to cause an increase in the equilibrium uvCSF volume relative to the equilibrium uvCSF volume present during the on-time segment of the cycle.

Further cycling of the compression from right to left, or left to right, will serve to fill the cranial veins from either the right or the left side and then potentially minimize, or even reverse, the direction of filling from the side being compressed. Then, as that first compression is released and the other side is compressed, a reversal of venous back flow (or reduction as one side of the venous forward flow ensues). The effect would be nearly a “to and fro”, or agitation, of the fluids, allowing for better mixing of volumes of blood or CSF with high concentrations of target substances with volumes of blood or CSF that are lower in concentration.

Thus, human subjects to which the methods and devices of this invention may be applied include, for example, athletes, soldiers, students, truck and car drivers, railroad engineers, pilots, astronauts, first responders (police, fire, EMTs), medical service providers (nurses and doctors), security guards, and industrial workers. Further, ailments that could be remedied include glaucoma, Meniere's Disease, and any of a number of neurodegenerative disorders such as Alzheimer's or Parkinson's.

It will be appreciated by persons having ordinary skill in the art that many variations, additions, modifications, and other applications may be made to what has been particularly shown and described herein by way of embodiments, without departing from the spirit or scope of the invention. Therefore, it is intended that scope of the invention, as defined by the claims below, includes all foreseeable variations, additions, modifications or applications.

The devices disclosed herein are therapeutic in nature and are used to treat maladies or diseases or aging rather than to prevent TBI or force impartations. The devices and methods disclosed herein are not attempting to fill a Compensatory Reserve Volume (CRV) to prevent hydrodynamic energy absorption (SLOSH), but rather are operable to effectuate flow throughout the fluid cranial space to include, but not be limited to, the arteries, veins, interstitial spaces (defining the glymphatic circulatory system), venous capacitance vessels, peri-lymphatic and endo-lymphatic fluids (auditory), ocular fluids (aqueous humor), Cerebrospinal fluids (CSF), and lacrimal fluids (tears). To maximize flow through the systems described above, the present disclosure contemplates compressing/occluding one side of the venous tree accessible by compressing each side of the neck in succession—one side, then the other side—and maintaining a frequency of compression then release to better allow a pumping action to maximize flow, rather than just a “filling” of the venous or CSF spaces. Specifically, in order to treat the conditions contemplated in the present disclosure, it is not beneficial to compress both sides of the venous vasculature tree simultaneously, as this would be like blowing on both ends of a straw which may pressurize or maximally fill an internal space but not create flow. Therefore, the devices and methods disclosed herein are configured and operable to compress or impede flow on one side, release pressure and then compress or impede flow on the other. The devices disclosed herein have the ability to compress just one vessel, or multiple vessels, on one side or the other, but not both sides of the neck at the same time.

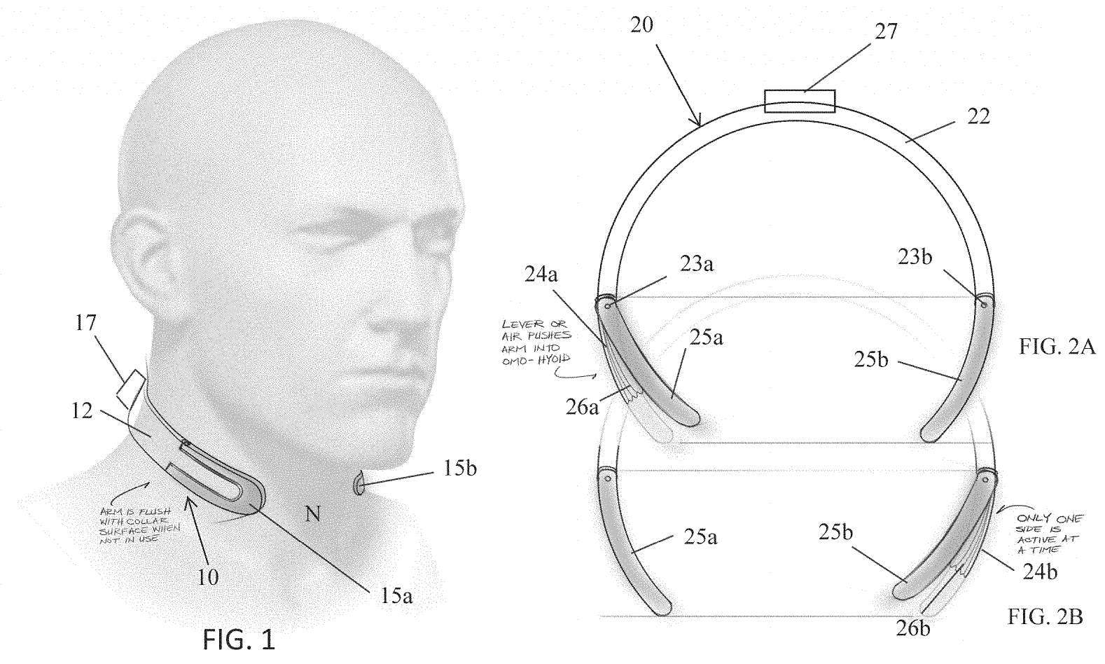

The present disclosure contemplates a compression device 10 having a fitted collar 12 with a compression element 15a, 15b at each end of the collar 12, as shown in FIG. 1. The device is sized in relation to the circumference of the neck N of the person so that the compression elements 15a, 15b are situated directly adjacent desired blood vessels of the neck. The device is configured as an open-C shape, rather than completely encircling the neck, to facilitate putting the device on and taking it off, and to ensure that the compressive force generated by the compression elements 15a, 15b is limited to the desired vessels. The fitted collar 12 can have a cylindrical cross-section, although a flattened cross-section can be preferable to more closely approximate the neck surface and to be less intrusive while lying down. The fitted collar is sufficiently rigid to provide a suitable base for pressure to be applied by the compression elements, yet still exhibit some resiliency to allow the collar to open slightly to be placed on the neck N of the subject. The inner face of the fitted collar 12 can be provided with a polymer panel or surface coating that allows the collar to “self-seat” and yet have enough friction to not slide on the neck of the subject.

A drive mechanism 17 is provided the middle of the collar at the back or closed portion of the open-C so that the mechanism is at the back of the neck of the subject. The drive mechanism includes an actuator and a power source that provides power to the actuator. The embodiments of FIGS. 2-10 contemplate different actuators, as described herein. The power source can be a battery or a socket for receiving power from an external power source, in which the external source can be electrical or pneumatic, depending on the nature of the actuator. The drive mechanism drives the compression elements 15a, 15b between neutral and compression positions, as described herein.

FIGS. 2A, 2B illustrate a compression device 20 with a fitted collar 22 configured like the collar 12. The collar 22 includes with a fulcrum or pivot point 23a, 23b at each end of the open-C that pivotably supports a corresponding lever 35a, 35b serving as the compression elements 15a, 15b. The collar 22 includes an extension plate 24a, 24b at each end that supports a bellows structure 26a, 26b disposed between the extension plate and the corresponding lever 25a, 25b. The bellows structures can be in the form of a small airbag, inflatable bladder, or the like. The drive mechanism 27 can include a pump or provide a connection to an external pump that is fluidly connected to each of the bellows structures 26a, 26b to provide pressurized air to each bellows structure causing the bellows to expand and push the corresponding lever outward about the pivot point 23a, 23b. The fluid connection between the drive mechanism and the bellows structures can be by tubes embedded within the fitted collar, or by a hollow interior of the collar. When the drive mechanism directs fluid to one of the bellows structures, the bellows structures pushes the associated lever into the skin to apply pressure to a vessel in the neck. In a preferred embodiment, the device 20 is positioned on the neck to that the levers can apply pressure to each external jugular vein, which resides atop the sternocleidomastoid muscle which resides over the omo-hypoid muscle which typically resides atop the internal jugular vein, on each side of the subject's neck N.

The drive mechanism 27 is configured to alternately inflate and deflate the bellows structures 26a, 26b of the device to alternately compress or impede venous blood flow on one side of the neck while simultaneously releasing the compression and permitting venous blood flow on the other side of the neck. The drive mechanism can incorporate a valve structure, such as a shuttle valve, between the fluid connections to each bladder structure, along with a controller that controls the valve structure to achieve the alternating pressure and release cycle for each lever 25a, 25b. The controller is configured to repeat the compression/release cycle over and over to effectively “pump” the volumes of venous blood back and forth to allow for better flow, and even turbulence as the venous blood stops and reverse direction as the corresponding side fills and engorges, only to then drain and deflate back and forth. The expanding force applied by each lever needs to be sufficient to overcome an internal pressure of approximately 3-5 mmHg and to overcome the stiffness of the overlying muscles and ligaments and skin. Typically, this force is in the range of 1-5 psi.

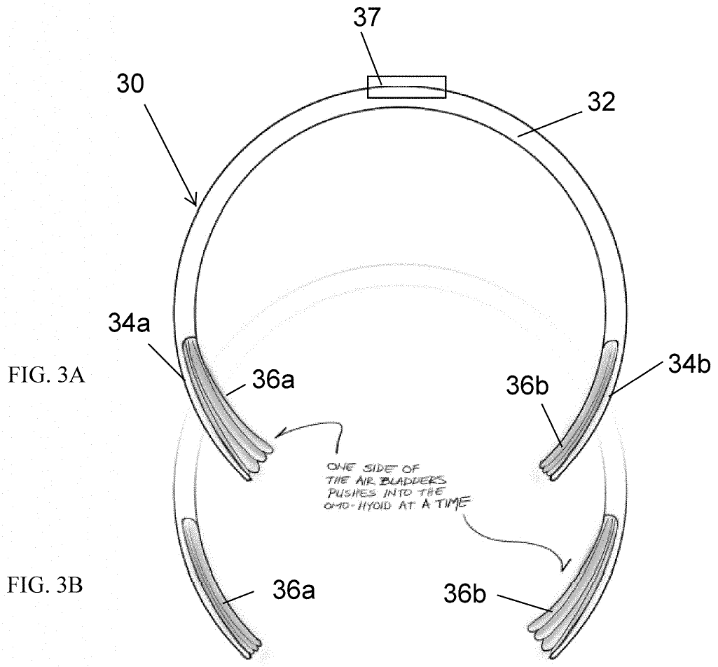

A second compression device 30, shown in FIGS. 3A-B, is similar to the device 20 but without the lever coming into contact with the skin. Thus, the device 30 includes a fitted collar 32 with a drive mechanism 37 at the back of the collar. The opposite ends of the collar include an extension plate 34a, 34b that carries corresponding bellows structures 36a, 36b, all similar to the extension plates and bellows structures of the device 20. For the compression device 30, the bellows structures 36a, 36b bear directly on the skin as first the left bellows 36a is expanded (FIGS. 3A) and then the right bellows 36b is expanded (FIG. 3B)

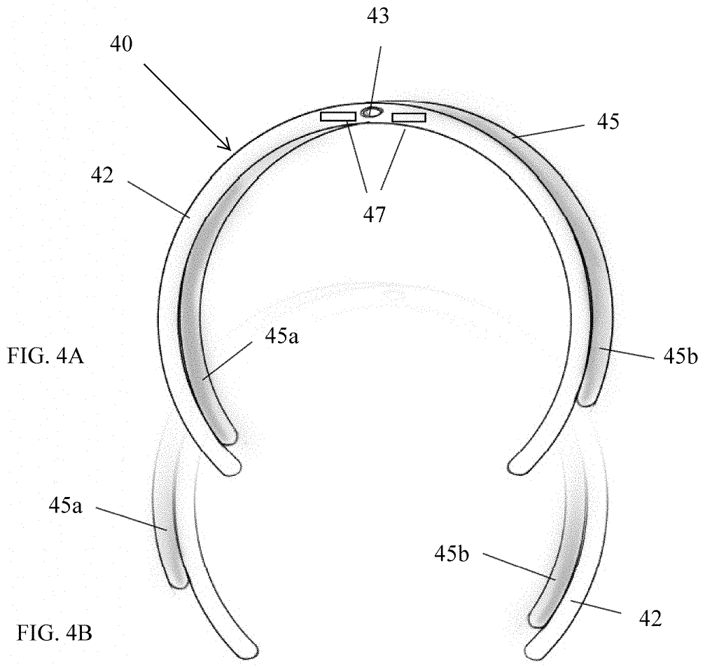

A third compression device 40, shown in FIGS. 4A-B, includes a C-shaped fitted collar 42 and a similarly C-shaped compression element 45 that is pivotably mounted to the fitted collar at a pivot point 43. The left and right arms 45a, 45b of the compression element 45 are configured to rock back and forth relative to the fitted collar worn on the neck of the subject, as shown in FIGS. 4A, 4B. The device 40 includes drive mechanism 47 configured to pivot the compression element 45 relative to the pivot point 43. In one specific embodiment, the drive mechanism 47 can include an electric motor, such as a stepper motor or servo-motor, configured to rotate an axle of the compression element at the pivot point or to push one side and then the other of the compression element on opposite sides of the pivot point 43. As with the previous embodiments, the drive mechanism is configured to alternately move the left and right arms 45a, 45b toward the neck of the subject to compress the external jugular veins on each side of the neck, wherein the compression is sufficient to impede venous blood flow through the vein.

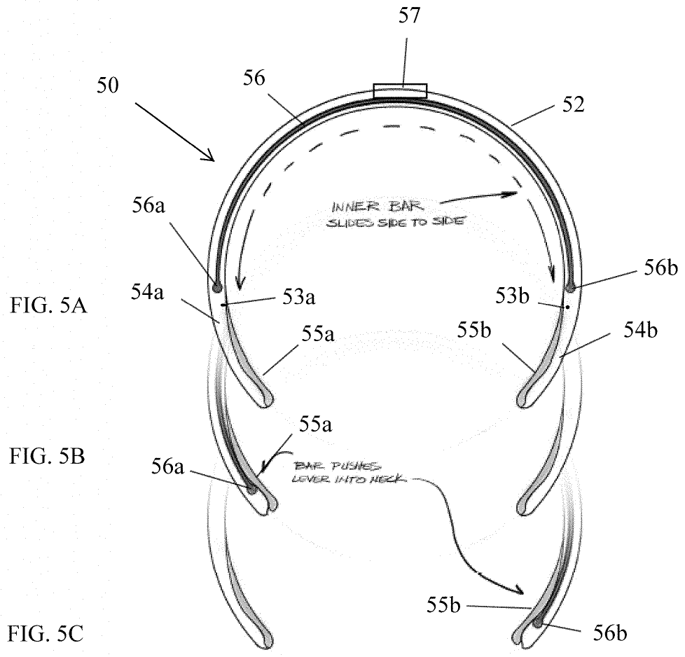

The compression device 50 shown in FIGS. 5A-C includes a fitted collar 52 with extension plates 54a, 54b at the left and right ends of the C-shape. Compression elements 55a, 55b are pivotably mounted at pivot points 53a, 53b to a respective extension plate. The compression elements 55a, 55b are in the form of curved plates adapted to bear against the neck of the subject. The device 50 includes an inner bar 56 that is slidably disposed within a hollow interior of the fitted collar. Each end of the inner bar includes an enlargement 56a, 56b, which can be in the form of a bulb, that bears against a respective compression element 55a, 55b as the bulb moves toward an end of the collar, as depicted in FIGS. 5B, 5C. The inner bar 56 starts at a neutral position, shown in FIG. 5A, in which the bulbs 56a, 56b are not in contact with the compression elements 55a, 55b. The drive mechanism 57 is operable to alternately move the inner bar 56 toward one extension plate 54a and then the other plate 54b, thereby alternately pushing the corresponding compression element 55a, 55b outward to apply pressure to the adjacent jugular vein of the subject. The drive mechanism 57 can incorporate a motor-driven mechanism, such as a rack and pinion or a lead screw, to move the inner bar 56 back and forth within the fitted collar 52.

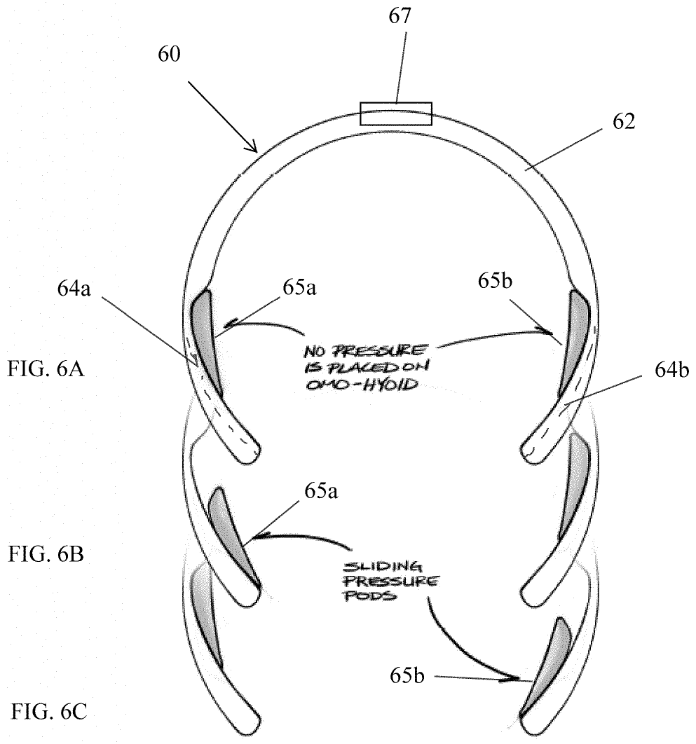

FIGS. 6A-C depict a fifth embodiment in which a compression device 60 includes a fitted collar 62 that defines sloped channels 64a, 64b at the opposite ends of the C-shape. Compression elements 65a, 65b are slidingly disposed within a corresponding channel so that each element sits in a neutral position, as shown in FIG. 6A, and is movable to a pressure position, as show in FIGS. 6B, 6C. The compression elements can be in the form of a wedge that presses against the subject's neck as it moves to the end of the corresponding channel 64a, 64b. The drive mechanism 67 is mechanically connected to each wedge compression element and is operable to alternately move each element 65a, 65b from the neutral position to the compression position.

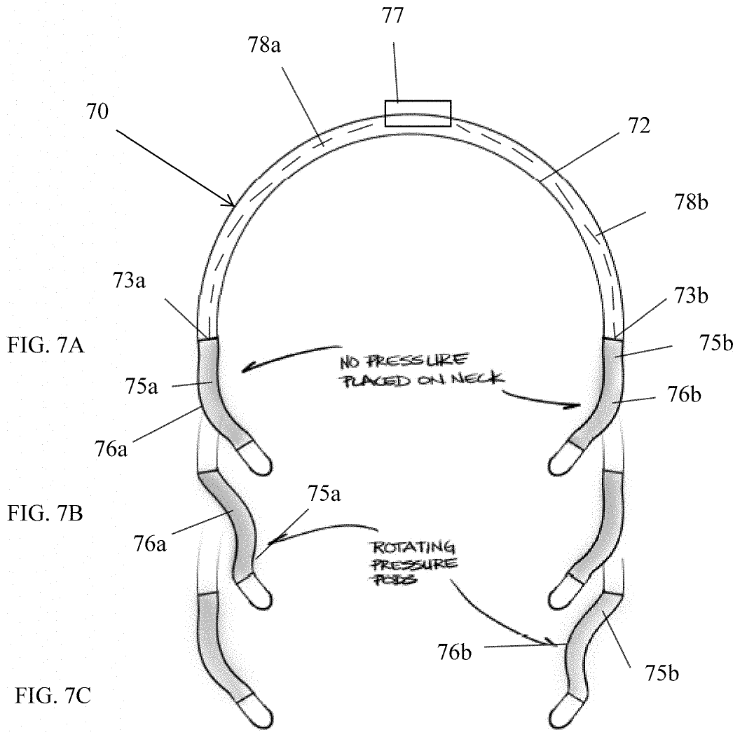

The compression device 70 in FIGS. 7A-C includes a fitted collar 72 with compression elements 75a, 75b rotatably mounted to the opposite ends 73a, 73b of the collar. The compression elements are bowed to provide a bend 76a, 76b at a midpoint of the compression elements. In the neutral position shown in FIG. 7A, the compression elements are arranged so that the bends 76a, 76b project outward away from the neck of the subject. The two compression elements are configured to rotate about the corresponding end 73a, 73b of the collar so that the bends 76a, 76b are directed inward toward the neck, as shown in FIGS. 7B, 7C. As with the devices of the previous embodiments, the drive mechanism 77 is configured to alternate the movement of the compression devices from the neutral (FIG. 7A) to the compress (FIGS. 7B, 7C) positions in with the bends 76a, 76b alternately compress the jugular veins at each side of the subject's neck. The drive mechanism can include an electric motor that drives a flexible rotating cable 78a, 78b fixed to each compression element 75a, 75b.

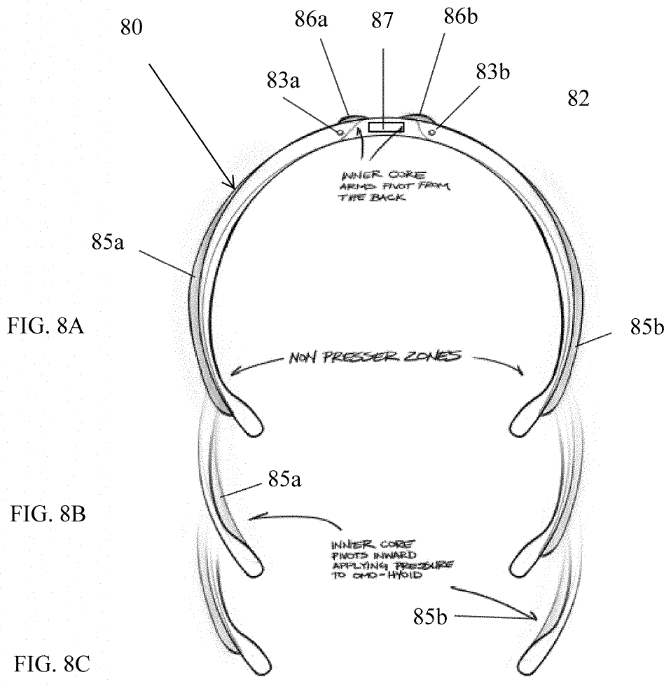

In a seventh embodiment, the compression device 80 of FIGS. 8A-C is similar to the device 40 except that the arms 85a, 85b are separate components that are pivotably mounted to the back face of the fitted collar 82 at respective pivot points 83a, 83b. The drive mechanism 87 is positioned between the two pivot points and is configured to pivot each arm 85a, 85b about the respective pivot point from the neutral position shown in FIG. 8A to the compress positions shown in FIGS. 8B, 8C. The drive mechanism 87 can include motor driven gears or pawls that engage the back ends 86a, 86b of the two arms to pivot the arms. As with the drive mechanisms of the other embodiments, the mechanism 87 can be configured to alternately pivot the arms inward at an adjustable frequency of the rocking motion to provide for left then right compression and release of the left and right jugular veins.

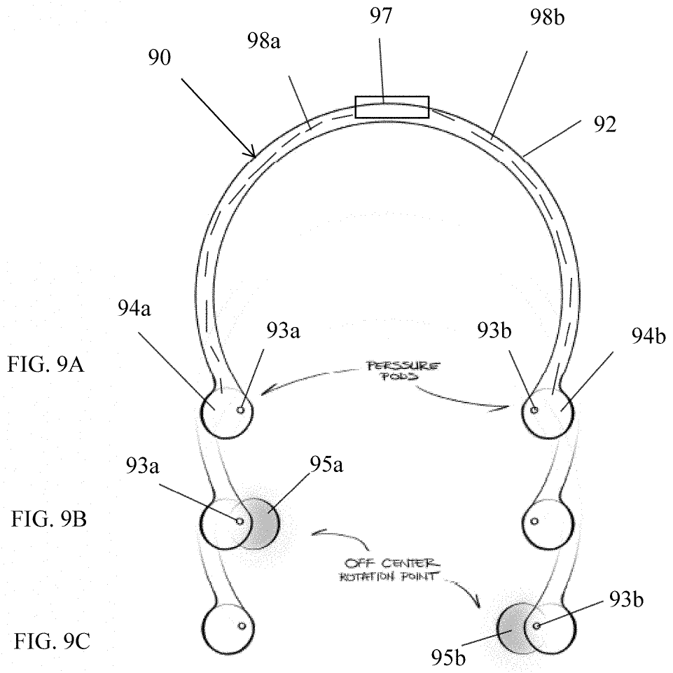

The compression device 90 shown in FIGS. 9A-C include a pair of rotating “pods” 95a, 95b that are pivotably mounted to the ends 94a, 94b of a C-shaped fitted collar 92. The pivot point 93a, 93b is offset from the center of the pods 95a, 95b so that the pods can pivot inward to apply pressure to the vessels in the subject's neck, as shown in FIGS. 9B, 9C. The drive mechanism 97 at the back of the collar 92 can include an electric motor that pushes or pulls a cable 98a, 98b connected to a respective one of the pods 95a, 95b. Pushing a cable toward an end of the collar pushes the corresponding pod, causing it to pivot about the offset pivot point 93a, 93b. In one specific embodiment, the cable can be continuous between the left segment 98a and the right segment 98b so that pushing the cable toward one end 94a, 94b of the collar necessarily retracts the cable from the opposite end, thereby ensuring that only one pod is compressing a vein at any particular time. The drive mechanism can incorporate a pinion on a rotary motor and a rack element on the continuous cable. As in the other embodiments, the drive mechanism includes a controller to alternately push the pods inward to compress the left, and alternately, the right jugular vein.

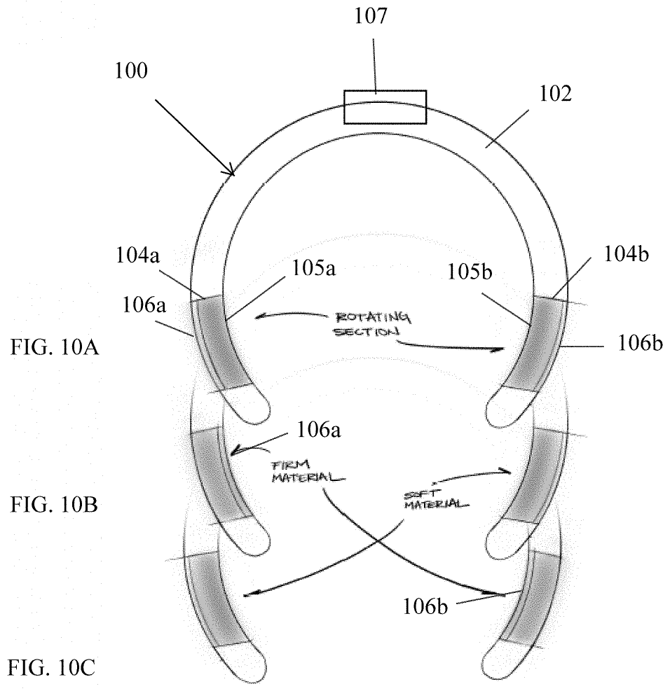

In a further embodiment, a compression device 100 illustrated in FIGS. 10A-C includes a fitted collar 102 with rotating sleeves 105a, 105b mounted at the respective ends 104a, 104b of the collar. The sleeves 105a, 105b are eccentrically configured, with a soft portion 105a, 105b facing the subject's neck in the neutral position of FIG. 10A and a more rigid portion 106a, 106b facing away from the neck in the neutral position. The sleeve is rotated in the compress position shown in FIGS. 10B, 10C so that the eccentrically disposed rigid portion 106a, 106b contacts the jugular region of the neck to compress the underlying vasculature. The drive mechanism 107 controls the rotation of the two sleeves 105a, 105b to alternate compression by the corresponding rigid portions 106a, 106b.

The compression devices disclosed herein are configured to alternate compression of vascular structures on opposite sides of a subject's neck to impact the intracranial pressure (ICP), intracranial volume (ICV). In particular, the devices are operable to increased ICP and/or ICV by diverting flow to the venous capacitance vessels of the brain by reducing the jugular venous outflow. Cranial venous flow may be diverted by applying pressure to one or more (e.g., one, two, three, four, or more) neck veins including, for example, the internal jugular vein(s) (“IJVs”) and the external jugular vein(s) (“EJVs”). This flow is then diverted to the venous capacitance vessels and vertebral veins and as the vessels dilate, the cerebral spinous fluid (CSF) equilibrates by circulating down through the interstitial and spinal canal spaces (defining the Glymphatic System). The compression elements, such as elements 25a, 25b of the device 20, for example, are sized and arranged to apply compression to one or more neck veins, depending on the desired treatment protocol.

The drive mechanisms of the compression devices, such as drive mechanism 27, for example, include a microcontroller, or similar device, that is configured to alternately and intermittently actuate one compression element, while releasing the other compression element to produce cyclic compression and releases. The microcontroller can be configured to apply these cyclic compressions and releases at a predetermined frequency, or at a selectable fast, moderate or slow frequency. In one embodiment, the fastest rate is measured in cycles per minute, the moderate rate is measured in cycles per hour and the slowest in cycles per day. The microcontroller of the drive mechanisms also controls the on-time of each cycle, where the on-time can be measured in seconds or in hours, again depending on the treatment protocol. Similarly, the off-time is controlled by the microcontroller, with off-times also measured in seconds or hours depending on the treatment protocol.

The drive mechanism and compression elements are configured to generate sufficient pressure against the neck of the subject to substantially occlude the desired venous structure. Thus, the compression devices disclosed herein are configured to generate occlusive pressures ranging from 10 mm Hg to 100 mm Hg.

The present disclosure provides methods for improving, repairing or preventing impairment of brain function in a subject by facilitating cerebral blood and cerebral spinal fluid flow. The brain function can be selected from the group consisting of arousal, attention or attentiveness, executive functions, learning, memory, motor coordination, spatial awareness, and vigilance. Repairing brain function would relate to improving the above functions after congenital malfunctions (such as hydrocephalus, normal pressure hydrocephalus, Arnold Chiari Malformation (types I, II, and III), spina bifida, syringomyelia, tethered cord syndromes, abnormalities of cortical development (Groups I, II, and III), vascular or cerebrospinal fluid (CSF) compartment abnormalities, or brain injuries. Brain injuries can be traumatic, closed head injuries, acceleration deceleration, contusions, penetrating or shaken baby-type vibrational injury, or acquired injuries or maladies such as toxic or anoxic brain damage (lack of oxygen), infectious or inflammatory conditions (i.e., meningitis or lupus cerebritis), stroke or tumor derived damage or malfunction. Toxic maladies include alcohol, hepatic encephalopathy, Wernicke-Korsakoff, Central Pontine Myelinolysis. Repairing brain function can also be required to address damage caused by any form of dementia such as Alzheimer's, Parkinson's, Pick's, Amyotrophic Lateral Sclerosis (Lou Gehrig's Disease), Vascular, Lewy Body, Frontal-temporal Dementia, Huntington's Disease, Creutzfeldt-Jakob Disease, or Mixed Dementia. The methods are also helpful to improve the above brain function in the setting of aging, ADHD, Post Traumatic Stress Disorder, and other psychiatric disorders such as, but not limited to insomnia, schizophrenia, bipolar disorder, or anxiety disorders. The methods disclosed herein can promote the delivery of endogenous chemicals (oxytocin, vasopressin, anti-diuretic hormone (ADH), or even exogenous chemical delivery such as nutrients, chemotherapy or inti-infectives whether they be anti-viral, anti-bacterial or anti-fungal.

The present disclosure also provides methods for reducing the concentration of somnogenic substances in the brain of a subject by increasing flow away from the brain and down the spinal cord through the Glymphatic Pathways. The methods include increasing intracranial pressure, blood volume, or both, by administering changes to inspired CO₂ levels by using exogenous or endogenous CO₂ with resultant pushing of CSF flow down from the cerebral compartments into the lower spinal canal and spinal bulb. Exogenous sources of CO₂ can include, but not be limited to, canister or reservoir sources of CO₂, or rebreathing of one's own CO₂ through the addition of various dead space volumes to the respiratory tract or alteration in positive pressure breathing (which alters ventilation).

In accordance with one feature of the present methods, the intracranial pressure, blood volume, or both can be increased by applying pressure to one or more neck veins, such as the internal jugular veins or at the external jugular veins. The neck vein pressure is about 10 mm Hg to about 100 mm Hg and is applied intermittently in a plurality of cycles, wherein each cycle is characterized as having an on-time having a first neck vein pressure and an off-time having a second neck vein pressure, wherein the first neck vein pressure is greater than the second neck vein pressure. The neck vein pressure is applied alternately to the right then left sides and then back again, at various predetermined frequencies and durations as per below. For example, the on-time can have a duration of 1 second to one hour and the off-time a duration of 1 second to one hour. The first neck vein pressure can be 10-100 mm Hg, while the second neck vein pressure can be 0-50 mm Hg. The intermittent pressure and release is cycled from one side to the other throughout the time period, creating a “to and fro” moving of cranial fluids.

What is claimed is:

1. A method for improving, repairing or preventing impairment of brain function in a subject by facilitating cerebral blood and cerebral spinal fluid flow, comprising increasing blood flow away from the brain and down the spinal cord and interstitial spaces through the Glymphatic Pathways.

2. The method of claim 1, further comprising increasing one or both of the intracranial pressure and the intracranial blood volume, to increase blood flow away from the brain.

3. The method of claim 2, wherein the step of increasing one or both of the intracranial pressure and the intracranial blood volume is achieved by administering changes to inspired CO₂ levels of the subject by using exogenous or endogenous CO₂.

4. The method of claim 2, wherein the step of increasing one or both of the intracranial pressure and the intracranial blood volume is achieved by applying pressure to one or more neck veins.

5. The method of claim 4, wherein the one or more neck veins includes at least one internal jugular vein or at least one external jugular vein.

6. The method of claims 4, wherein pressure applied to the one or more neck veins is about 10 mm Hg to about 100 mm Hg.

7. The method of claim 4, wherein the pressure applied to one or more neck veins is applied intermittently in a plurality of cycles, wherein each cycle is characterized as having an on-time having a first neck vein pressure and an off-time having a second neck vein pressure, wherein the first neck vein pressure is greater than the second neck vein pressure.

8. The method of claim 7, wherein the on-time has a duration of one second to one hour.

9. The method of claim 7, wherein the off-time has a duration of one second to one hour.

10. The method of claim 7, wherein the first neck vein pressure is 10-100 mm Hg.

11. The method of claim 7, wherein the second neck vein pressure is 0-50 mm Hg.

12. The method of claim 7, wherein the first neck vein pressure is at least 20 mm Hg greater than the second neck vein pressure.

13. The method of claim 7, wherein each cycle has a duration of five seconds to two hours.

14. The method of claim 4, wherein the pressure applied to one or more neck veins is applied alternately to one or more veins on one side of the neck and then to one or more veins on the other side of the neck.

15. A method for improving, repairing or preventing impairment of brain function in a subject by facilitating cerebral blood and cerebral spinal fluid flow by increasing blood flow away from the brain and down the spinal cord through the Glymphatic Pathways, comprising applying pressure to one or more neck veins on each side of the subject's neck, wherein the pressure is applied alternately to one or more veins on one side of the neck and then to one or more veins on the other side of the neck.

16. The method of claim 15, wherein the pressure is applied intermittently in a plurality of cycles, wherein each cycle is characterized as having an on-time having a first neck vein pressure and an off-time having a second neck vein pressure, wherein the first neck vein pressure is greater than the second neck vein pressure.

17. The method of claim 16, wherein the on-time has a duration of one second to one hour.

18. The method of claim 16, wherein the off-time has a duration of one second to one hour.

19. The method of claim 16, wherein the first neck vein pressure is 10-100 mm Hg.

20. The method of claim 16, wherein the second neck vein pressure is 0-50 mm Hg.

21. The method of claim 16, wherein the first neck vein pressure is at least 20 mm Hg greater than the second neck vein pressure.

22. The method of claim 16, wherein each cycle has a duration of five seconds to two hours.

23. A device for applying pressure to one or more neck veins on each side of the neck of a subject, comprising:
a circumferential C-shaped collar sized to partially encircle the neck of the subject;
a pair of compression elements movably mounted on the collar, each compression element sized and oriented on said collar to apply pressure to one or more neck veins on each side of the neck; and
a drive mechanism for moving said compression elements relative to said collar to alternately apply pressure to the one or more neck veins on each side of the neck of the subject, wherein the pressure is sufficient to restrict blood flow egressing from the head of the subject through the one or more neck veins.

24. The device of claim 23, wherein:
said C-shaped collar includes opposite ends: and
each of said pair of compression elements is movably mounted to a corresponding one of said opposite ends.

25. The device of claim 24, wherein each of said pair of compression elements is a lever pivotably or slidably mounted to said corresponding one of said opposite ends of said collar.

26. The device of claim 24, wherein each of said pair of compression elements is an expandable bellows structure mounted to said corresponding one of said opposite ends of said collar.

27. The device of claim 23, wherein said pair of compression elements are at opposite ends of a C-shaped compression element that is pivotably mounted to said C-shaped collar to pivot between a neutral position in which said C-shaped compression element is overlapped by said C-shaped collar, and compression positions in which one or the other of the opposite ends of said C-shaped compression element is pivoted toward the neck of the subject.
